(19) United States
(12) Patent Application Publication
(10) Pub. No.: US 2019/0347567 A1
(43) Pub. Date: Nov. 14, 2019

(54) METHODS FOR DATA SEGMENTATION AND IDENTIFICATION

(71) Applicant: Genetic Intelligence, Inc., New York, NY (US)

(72) Inventors: eMalick G. Njie, Brooklyn, NY (US); Bertrand Adanve, New York, NY (US)

(21) Appl. No.: 15/919,416

(22) Filed: Mar. 13, 2018

(51) Int. Cl. G06N 20/00; G06F 16/23; G06N 3/08; G06N 3/04; G16B 40/30; G16B 20/20

(52) U.S. Cl. CPC G06N 20/00; G06F 16/2365; G16B 20/20; G06N 3/04; G16B 40/30; G06N 3/08

(57) ABSTRACT

The present invention provides tools, systems and methods for identification of an unknown object through recognition and matching of its underlying data against a previously segmented relevant data set. Specifically, the present invention provides methods for data segmentation and identification comprising applying nonlinear dimension reduction of data, and preferably artificial intelligence (“AI”) based nonlinear dimension reduction of data, followed by comparison and identification.

### FIELD OF THE INVENTION

This invention relates to systems and methods for segmenting data and identifying new subject data.

### BACKGROUND OF THE INVENTION

In the following discussion certain articles and processes will be described for background and introductory purposes. Nothing contained herein is to be construed as an “admission” of prior art. Applicant expressly reserves the right to demonstrate, where appropriate, that the articles and processes referenced herein do not constitute prior art under the applicable statutory provisions.

In many fields, the ability to accurately identify and/or distinguish the position of an object is a sought-after and valuable capability for machine systems. The data underlying the objects can be used to achieve this outcome by employing a historical data set to successfully parse and describe a certain number of objects, followed by matching of future unknown objects to the established historical objects. The neuronal systems of human beings employ such an approach to learn, distinguish, remember, and identify the objects that they encounter throughout life, be they living or inert. Endowing machine systems with the abilities to learn, distinguish, remember, and identify objects at various scales may allow such systems to formulate better decisions. For instance, an artificial intelligence (“AI”) system used for data segregation and identification in a self-driving vehicle that can successfully recognize and learn from the objects it encounters can make better future decisions as an aid to successfully reaching destinations.

There is thus a need for more accurate and defined analysis tools for stratification of complex data sets. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings, and as set forth in the examples and appended claims.

The present invention provides tools, systems and methods for identification of an unknown object through recognition and matching of its underlying data against a previously segmented, relevant data set. Specifically, the present disclosure provides methods for data segmentation and identification comprising applying two sequential methods: 1) a first method that segregates data, e.g., by clustering the data using nonlinear dimension reduction of data, and preferably using artificial intelligence (“AI”)-based nonlinear dimension reduction of data; and 2) a method that memorizes the positions of the data segregated using the first method, compares new input data with the memorized positions, and identifies the new data with respect to the memorized features. The combination of the segregation (e.g., dimension reduction) techniques and the machine learning-based identification techniques as provided herein are particularly well suited for the study of complex, high-dimensional datasets.

The systems and methods of the present disclosure can be implemented for various uses involving complex data sets, including, but not limited to identification of genetic heritage in populations of admixed individuals; healthcare applications, such as diagnosis of different cancer types and/or stages and identification of patient populations with responses to potential therapeutic modalities; financial instruments and/or markets, .and practical object recognition, e.g., by autonomous vehicles or other machines that benefit from autonomous learning.

An exemplary, simplified work flow for specific systems and methods of the disclosure is illustrated in FIG. 1. Various specific features that may be added to the work flow as illustrated, e.g., optional feedback, are described in more detail herein and are not shown for clarity.

Accordingly, in certain aspects, the disclosure provides a system for data segmentation and identification, comprising a first tool for segregation of complex data sets; and a second tool for memorization of segregated data and identification of new data by comparison to the memorized data.

In certain preferred aspects, the segregation of complex data sets uses non-linear dimension reduction. In specific aspects, the nonlinear dimension reduction uses an AI-based non-linear dimension reduction tool, e.g., t-distributed stochastic neighbor embedding (“t-SNE”).

In other preferred aspects, the system uses an AI-based tool for memorization of segregated data and identification of new data by comparison to the memorized data. In preferred aspects, the system for memorization of segregated data and identification of new data by comparison to the memorized data uses an artificial neural net.

In certain aspects, the disclosure provides a method for identification of data from one or more individuals, comprising inputting a plurality of data points into a tool for nonlinear dimension reduction, applying nonlinear dimension reduction to the data points to segment the data into clusters, inputting the segmented data into an AI-based tool, inputting one or more individual data points into the AI-based tool comprising the segmented data, comparing the data from the one or more individual data points to the segmented, clustered data; and identifying the one or more individual data points by correlation with the segmented data memorized within the AI-based tool.

Accordingly, in certain aspects, the methods use a first tool for segregation and/or identification of complex data sets for data segmentation and identification, and a second tool for memorization of segregated data and identification of new data by comparison to the overall memorized data. In specific aspects, the nonlinear dimension reduction of the methods uses an AI-based non-linear dimension reduction tool, e.g., t-distributed stochastic neighbor embedding (“t-SNE”).

In other preferred aspects, the methods use an AI-based tool for memorization of segregated data and identification of new data by comparison to the memorized data. In preferred aspects, the methods for memorization of segregated data and identification of new data by comparison to the memorized data use an artificial neural net.

In other preferred embodiments, the methods allow for identification of new data through separate or concurrent segmentation of the new data together with the overall data using non-linear dimension reduction and subsequent memorization and identification by the second AI-based tool. In specific aspects, the features of the new data can first be extracted in relation to the segmented clusters, and the extracted relational features are then analyzed by the second AI-based tool.

In certain implementations, the systems and methods of the disclosure can utilize a plurality of data points are on objects for use with autonomous vehicle performance. In other implementations, the methods of the disclosure utilize a plurality of data points on financial instruments and/or markets.

In specific implementations, the systems and methods of the disclosure are used for genetic and/or healthcare purposes. Accordingly, in certain implementations, the systems and methods of the disclosure can utilize a genetic data used for population stratification.

In other implementations, the data points are data points are used to determine the presence and/or concentration of microbial organisms or virus, e.g., genetic or other data that are indicative of specific bacterial, fungal or viral pathogens.

In yet other implementations, the input data points are used to determine the predicted response of one or more individuals to a particular therapeutic intervention, e.g., determining the best therapeutic modality based on identification of biomarker, determining metabolism of a drug based on genotype, identifying cell surface markers for treatment of various cancers, and the like. In still other implementations, the input data points are used to determine the presence and/or stage of a disease in one or more individuals.

In related implementations, the genetic data points can be used to determine the genetic heritage of one or more individuals. In specific implementations, the disclosure provides methods for including or excluding one or more individuals as being descended from a particular heritage, comprising the steps of inputting data from the genetic data of a plurality of individuals into a tool for nonlinear dimension reduction, applying nonlinear dimension reduction to the data from a plurality of individuals to segment the data into clusters, inputting the segmented genetic data into an artificial intelligence (“AI”)-based tool, inputting genetic data from one or more individuals into the AI-based tool comprising the segmented genetic data, comparing the genetic data from one or more individuals to the segmented genetic data from the plurality of individuals, and including or excluding one or more individuals as being descended from a particular heritage by identifying the correlation of the individual data with segmented genetic data within the AI-based tool.

The systems and methods of the disclosure can further utilize a data correction tool. This tool can be applied at various stages of analysis. For example, the data correction tool can be a relatedness analysis tool that is applied to the data before the data is entered into the dimension reduction tool. In another example, the data correction tool can be utilized after the segregation tool to better delineate the results, e.g., an artificial neural net. In certain aspects, two or more data correction tools can be used at various stages in a work flow system.

The system of the disclosure, as illustrated generally in FIG. 1, can optionally comprise a feedback loop throughout the system. This optional feedback loop can be implemented in the system by use of a circuit of one or more controller AI tools that modulate one or more of the principal AI tools in the system,

In specific aspects, the present disclosure provides a multi-layered platform that inputs hyper-dense SNP maps composed of several million dimensions into the unsupervised machine learning paradigm, t-distributed stochastic neighbor embedding (t-SNE). t-SNE is a technique that is particularly well-suited for embedding high-dimensional data into a space of two or three dimensions, which can then be visualized in a scatter plot. Specifically, t-SNE models each high-dimensional object by a lower dimensional point (e.g., two- or three-dimensions) in such a way that similar objects are modeled by nearby points and dissimilar objects are modeled by distant points.

These and other aspects, features and advantages will be provided in more detail as described herein.

### BRIEF DESCRIPTION OF THE FIGURES

FIG. 1 is a schematic diagram illustrating a simplified view of the system.

FIG. 2 is a schematic view illustrating the segregation of heritages of individuals using principal component analysis (PCA).

FIG. 3 is a schematic view illustrating the segregation of heritages of individuals in 2d t-SNE.

FIG. 4 is a schematic view illustrating the segregation of heritages of individuals in 3D t-SNE.

FIG. 5 is a graphical depiction of a 5-layer neural net constructed to accept the t-SNE feature space of the 1000 genomes individuals.

FIG. 6 is a graph showing top-1 error rates by the number of dimensions employed with t-SNE and evidences a certain threshold (e.g., 4D) beyond which improvements are no longer optimal.

FIGS. 7A through 7E illustrate predictions of 26 labels of 2D through 6D t-SNE, respectively, shown as normalized confusion matrices.

FIGS. 8A and 8B are histograms of the firings of the neurons sensitive to a particular heritage.

FIGS. 9A and 9B depict respectively a schematic view of data augmentation technique and the described perplexity usage to achieve data augmentation by superposition.

FIGS. 10A and 10B are bar graphs depicting top-1 to top-5 error scores for 2D, 3D, 4D, 5D and 6D t-SNE without superposition (10A) and with superpositions (10B).

FIG. 11 shows contour plots of neural net activations.

FIG. 12 shows the distribution of population-wide top firing neuron intensities at various t-SNE dimensions and runs.

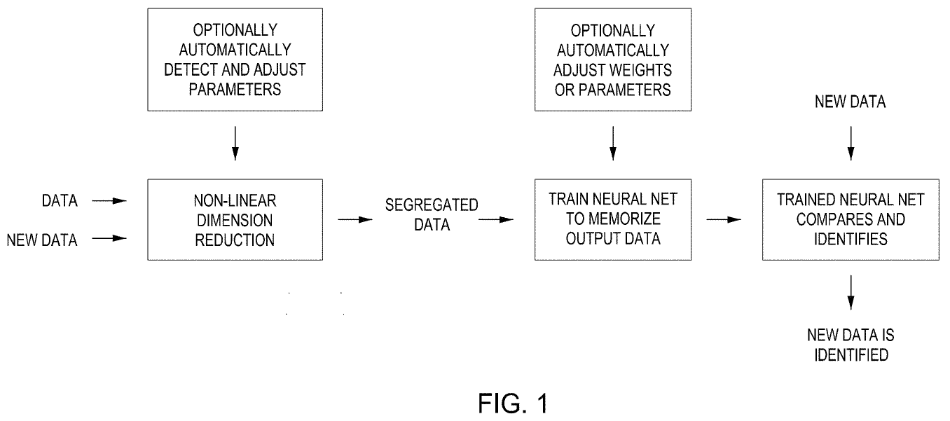

### DETAILED DESCRIPTION OF THE INVENTION

The following description is presented to enable one of ordinary skill in the art to make and use the invention and is provided in the context of a patent application and its requirements. Various modifications to the exemplary embodiments and the genetic principles and features described herein will be readily apparent. The exemplary embodiments are mainly described in terms of particular processes and systems provided in particular implementations. However, the processes and systems will operate effectively in other implementations. Phrases such as “exemplary embodiment”, “one embodiment” and “another embodiment” may refer to the same or different embodiments.

The exemplary embodiments will be described with respect to methods and compositions having certain components. However, the methods and compositions may include more or less components than those shown, and variations in the arrangement and type of the components may be made without departing from the scope of the invention.

The exemplary embodiments will also be described in the context of methods having certain steps. However, the methods and compositions operate effectively with additional steps and steps in different orders that are not inconsistent with the exemplary embodiments. Thus, the present invention is not intended to be limited to the embodiments shown, but is to be accorded the widest scope consistent with the principles and features described herein and as limited only by appended claims.

It should be noted that as used herein and in the appended claims, the singular forms “a,” “and,” and “the” include plural referents unless the context clearly dictates otherwise. Thus, for example, reference to the effect of “a neuron” may refers to the effect of one or a combination of neurons, and reference to “a method” includes reference to equivalent steps and processes known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and processes that are described in the publication and which might be used in connection with the presently described invention.

Those skilled in the art will recognize, or be able to ascertain using no more than routine experimentation, many equivalents to the specific embodiments of the invention described herein in the detailed description and figures. Such equivalents are intended to be encompassed by the claims.

For simplicity, in the present document certain aspects of the invention are described with respect to use of certain methods. It will become apparent to one skilled in the art upon reading this disclosure that the invention is not intended to be limited to a specific use, and can be used for in a wide array of implementations including identification of genetic heritage in the populations of admixed individuals, healthcare uses including diagnosing cancer types and/or stages and identifying the utility or patient response to potential therapeutic modalities, and, practical object recognition, e.g., by autonomous vehicles.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The terms “artificial intelligence” and “AI” as used interchangeably herein denotes intelligent systems or techniques such as machine learning, artificial neural networks, and the like.

An artificial neural network or “ANN” is a computational model based on the structure and functions of biological neural networks. Information that flows through the network affects the structure of the ANN because a neural network changes based on data that is inputted and/or outputted to the network.

The term “convolutional artificial neural network” or “CANN” as used interchangeably herein refers to a multilayered, interconnected neural unit collection in which the neural unit processes a portion of receptive fields (e.g., for inputting images). CANNs can be based on a computational algorithmic architecture in which the connectivity patterns between the neural units model the analytical processes of the visual cortex of the brain in processing visual information. The neural units in CANNs are generally designed and arranged to respond to overlapping regions of the receptive field for image recognition with minimal amounts of preprocessing to obtain a representation of the original image. CANNs can utilize reconfigurations of component parts (e.g., hidden layers, connections that jump between layers, etc.) to improve representations of the input data. One example of CANN construction can be found in Krizhevsky et al., ImageNet Classification with Deep Convolutional Neural Networks Advances in Neural Information Processing Systems 25 (NIPS 2012).

The term “genetic features” as used herein includes any feature of the genome, including sequence information, epigenetic information, etc. that can be used in the methods and systems as set forth herein. Such genetic features include, but are not limited to single nucleotide polymorphisms (“SNPs”), insertions, deletions, codon expansions, methylation status, translocations, duplications, repeat expansions, microsatellites, rearrangements, copy number variations, multi-base polymorphisms, splice variants, etc.

The term “nonlinear dimension reduction” refers to a method for reducing data dimensionality in a nonlinear fashion for understanding and/or visualizing the structure of complex data sets.

“Nucleic acid sequencing data” as used herein refers to any sequence data obtained from nucleic acids from an individual. Such data includes, but is not limited to, whole genome sequencing data, exome sequencing data, transcriptome sequencing data, cDNA library sequencing data, kinome sequencing data, metabolomic sequencing data, microbiome sequencing data, single nucleotide polymorphism determination and the like.

An “AI-based dimension reduction method” for use in the present disclosure includes any dimensionality reduction method that is particularly well suited for the segregation and/or visualization of high-dimensional datasets, e.g., datasets of genomic features.

“t-Distributed Stochastic Neighbor Embedding” or “t-SNE” is a nonlinear, AI-based dimension reduction method that allows for visualization of high dimensional data by giving each data point a location in a map, e.g., a two or three-dimensional map. Modifications can also allow for inclusion of additional useful dimensions (e.g., 4D, 5D, etc. vide infra).

### The Invention in General

The present invention provides systems, tools and methods for segmenting data, e.g. into relevant clusters and/or categories, against which the identity of new subject data can be easily determined. The invention includes a combination of non-linear dimension reduction with comparison and identification of new data using machine learning techniques, e.g., using an artificial neural network. In specific aspects, the systems and methods of the disclosure utilize a combination of AI-based non-linear dimension reduction and a machine learning method that memorizes segregated data and compares and identifies the position of new data (e.g., an artificial neural net) for stratification of complex data sets. This combination of tolls is a novel approach broadly applicable to any number of data, especially those of high dimensions, and including genetic sequences

Dimension reduction is the process of converting a set of complex data sets into data with lesser dimensions while still conveying the data in a more tractable manner. Traditionally in machine learning, dimension reduction methods (e.g., t-SNE) are used to obtain better features for a classification or regression task. These techniques are conventionally used to modify the data coming from machine learning systems such as artificial neural nets, e.g. post-hoc applications to analyze the activations of neurons within neural nets to see how they learned (Ossa H et al., PLoS One. 2016; 11(10):e0164414).

An important aspect of the invention is that the methods of the disclosure utilize dimension reduction tools in a unique manner. Rather than using dimension reduction following the analysis of data in, e.g., an artificial neural network, the methods of the present disclosure use an inverse approach, first applying the nonlinear dimension reduction system (e.g., t-SNE) and subsequently using the output of this method as the input for training of an artificial neural network. The neural nets used in the systems and methods of the invention therefore take Cartesian coordinates of t-SNE space (among other usable features) and convert this input into in an easily readable and accurate neuronal firing pattern.

In a specific aspect, the disclosure provides an AI-based nonlinear dimension reduction method such as t-SNE followed by an artificial neural network that learns the re-imagined t-SNE space as a novel map to accurately segregate and identify/distinguish data sets.

In another specific aspect, the cluster or classification scores from the dimension reduction algorithm are connected to one or more AI algorithm to automatically identify the parameters and/or hyperparameters to achieve the best clustering results and thus best new data object identification results by the final AI. In this aspect, one set of algorithms performs a clustering task and the other tells it whether its performance is good; if the performance is sub-par, the set of algorithms changes parameters and hyperparameters until their performance in comparing and identifying the data as determined by the other set of algorithms is considered acceptable for the desired use. For instance, the function of the additional set of AIs in this feedback loop can be performed by a generative adversarial neural net.

In another aspect, the new object data identification scores of the final output are fed into the feedback loop of the third AI set to confirm performance of the algorithms.

In another aspect, both the clustering scores of the dimension reduction step and dissection scores of the identification/final step are fed into the feedback loop of the third AI set to confirm performance of the algorithms.

In yet another specific aspect, the output of a relatedness analysis is fed into a nonlinear dimension reduction system such as an AI-based dimension reduction (e.g., t-SNE).

In some aspects, the present disclosure provides a multi-layered platform for inputting hyper-dense data maps of several million dimensions into an unsupervised machine learning system, e.g., t-SNE. By reconfiguring the t-SNE space as a novel cartographic map in genetic language, coordinates of this space were inputted into a neural net to allow the neural net to memorize the input from the t-SNE analysis, and compare new data with the memorized input to identify the position of new data relative to the memorized data. The firings of output neurons, each specific to a defined class of data, can be used for comparison and identification of complex data sets.

The successful performance of the invention on validation data on which it was not trained implies little to no overfitting, allowing the system to be scaled further to additional dimensions at the dimension reduction step. The output of the dimension reduction AI is a feature space that, if needed, can be visualized by human eyes and optimized for further improvements.

### Determination of Genetic Heritage

Existing methods for genetic ancestry inference are limited by several challenges, including the representative power of the input genetic data (e.g. type, distribution and number of markers), the populations designated as references (i.e., ground truth), and the power of the statistical methods employed to analyze the data.

Currently, the majority of direct-to-consumer tests for genetic ancestry rely on lineage-based, haploid, uniparental markers such as mitochondrial DNA (mtDNA) or Y-chromosome markers, which are maternally or paternally inherited (respectively) without recombination (Royal C D et al., American journal of human genetics. 86(5):661-73 (2010)). However, these account for only a small, unrepresentative fraction of an individual's total genetic ancestry and cannot be used to determine biogeographical ancestry. For instance, a similar mtDNA match between an individual in Africa and an individual in Asia can indicate that they share a common distant ancestor, but whether the African individual has recent Asian heritage, or the Asian individual has recent African heritage cannot be determined.

A recent study showed that estimates of continental ancestry vary widely between individuals with the same mtDNA haplogroup (Emery L S et al. American journal of human genetics. 96(2):183-93 (2015)). Autosomal markers can provide more information on individual ancestry since they cover a greater proportion of the genome, but these are still limited as each genomic segment in an individual represents only a small fraction of ancestors, and not every ancestor passes on his/her DNA at any given genomic segment. A more comprehensive approach is low-density genome-wide genotyping arrays. However, these suffer from ascertainment biases due to the inclusion of SNPs that are common only in a select subset of populations (Hellenthal G et al., Science. 343(6172):747-51 (2014); Albrechtsen A, et al., Molecular Biology and Evolution. (2010); 27(11):2534-47; Lachance J and Tishkoff S A. BioEssays: news and reviews in molecular, cellular and developmental biology, 35(9):780-6 (2013); Pritchard J K and Stephens M, Donnelly P. Genetics. 155(2):945-59 (2000); Tang H, et al., Peng J et al. Genet Epidemiol, 28(4):289-301 (2005). Ancestry informative markers (AIMs) can be used to estimate the geographical origins of the ancestors of an individual, but this is limited largely to pre-selected, broad continental regions (e.g. Africa, Asia, Europe or Native American), and do not provide sub-continental level resolution.

These methods would benefit from more powerful tools and approaches of the present disclosure that have the ability to take advantage of the vast amount of whole genome sequencing data made available by current sequencing technology (Gudbjarts son D F et al., Nat Genet. 47(5):435-44 (2015); England G. Genomics England and the 100,000 Genomes Project. UK (2013)).

The system of the invention utilizes a combination of a method for segregation of complex data sets (e.g., non-linear dimension reduction) and a machine learning method for memorization of segregated data and comparison and identification of new data with respect to the memorized data (e.g., an artificial neural net) to determine the genetic heritage of an admixed individual's genetic heritage or a population of individuals of a common heritage.

In the context of genealogical studies based on genetic information, “genetic admixture” occurs when individuals from two or more separate populations begin producing offspring, and the resulting descendants are referred to as “admixed.” Amongst the surprising results of the sequencing of large numbers of human genomes is the admixed nature of Homo sapiens.

Admixture analysis is a central problem in human genomics. In studies to identify the genetic causes of various phenotypes, it is important to control for population difference effects since true disease-causing mutations can be masked by subtle differences in genetic ancestry between case and control groups (Tian C et al., Hum Mol Genet. 2008; 17(R2):R143-50). Admixture analysis is also widely used to help people better understand their family history and identity (Hellenthal G, et al., Science, 2014; 343(6172):747-51). Existing methods of admixture analysis would benefit from more powerful tools and approaches to take advantage of the vast amount of genetic data made available by current sequencing technology.

More conventional genetics-based ancestry estimation tools are capable of analyzing an admixed individual's genome, comparing the individual's genome with reference models corresponding to various geographical regions, and determining percentages of the individual's genome that are inherited from ancestors from specific geographical regions. Many other methods of admixture analysis focus on the use of SNP genotyping chips, which are usually designed for a particular population. The application of such chips to different populations around the world can result in SNP ascertainment bias, which can distort measures of human diversity (Albrechtsen et al, 2010; Lachance and Tishkoff, 2013). Thus, the present tools, systems and methods of the present disclosure capable of analyzing unbiased SNPs derived from whole genome sequencing provide more accurate measures of admixture levels.

A particular challenge in determining ancestry is tracing ancestries associated with the smaller percentages. Given the ancestry proportion estimates, an individual may wish to know at what point in his or her heritage a full-blooded ancestor from a specific geographical region was introduced into the admixture.

### Dimension Reduction Tools for Complex Data

The aim of dimension reduction is to transform high dimensional problems into a visually tractable form. For instance, in genetics, the Homo sapiens genome is composed of some 3 billion characters that in the imagination of many geneticists form a linear string. A non-linear dimension reduction viewpoint would rephrase this as at least three billion dimensions, and since humans have difficulty making sense of more than three dimensions, reduction of more than three billion dimensions to two or three is a sensible means to more insightfully understand the genome.

In a particular embodiment, the nonlinear dimension reduction tool used is the unsupervised machine learning technique t-SNE, which aims to preserve structure of high dimensional data in a low dimensional map using spring/repellent laws of physics (van der Maaten L and Hinton G, Journal of Machine Learning Research, 9: 2579-2605 (2008)). t-SNE aims to minimize the Kulback-Leibler divergence between a Gaussian distribution of the original high dimensional data and a Cauchy distribution of the low dimensional representation by applying gradient descent, which helps to preserve the local structure of the data while revealing global structures in the form of clusters (van der Maaten L and Hinton G, Journal of Machine Learning Research, 9: 2579-2605 (2008)).

A final low-dimensional embedding can be obtained by applying gradient descent to iteratively minimize the Kulback-Leibler divergence between the two distributions. Data points corresponding to individuals can be represented as an interconnected web of springs, and the stiffness of a spring connecting any two individuals is determined by their genetic relatedness. Thus, within a dynamic system individuals related to each other attract to clusters while unrelated individuals repel each other thus creating space between clusters until equilibrium is reached. The result of t-SNE is both a local and global structure at the genome level reduced to a low-dimensional map that is visually accessible by humans (Li W et al., J. Bioinform Comput Biol 15:417500-17 (2017).

Other non-linear dimension reduction tools can also be used in the present disclosure, e.g., Isomap ((Tenenbaum, de Silva and Langford, Science 2000); Locally Linear Embedding (Rowels & Saul, Science 2000); Local Tangent Space Alignment (Zhang and Zha arXiv 2002); MDS (Bronstein, Kimmel et al., PNAS 2006); Random Trees (Ho, Proceedings of the 3rd International Conference on Document Analysis and Recognition (1995)); parametric t-SNE (van der Maaten, Learning a Parametric Embedding by Preserving Local Structure. Proceedings of the 12th International Conference on Artificial Intelligence and Statistics (AISTATS) Vol. 5 of JMLR: W&CP 5(2009)).

### Relatedness Analysis Tools

Before entering the segregated data into the dimension reduction tool, the segregated data may be subject to certain “quality control” aspects such as the performance of relatedness analysis, e.g., confirming the origin and individuality of data input into the systems of the disclosure. Various tools can be utilized, for example to confirm that data originated from the expected individual. For example, tools such as PLINK (Purcell, Set al., Am. J. Hum. Genet., 81:559-575 (2007) and KING (Manichaikul A et al., Bioinformatics; 26(22):2867-73(2010)) can detect sex and pedigree errors. In another example, a method such as PEDDY (Pedersen and Quinlan, The American Journal of Human Genetics 100:3, p406-413 (2017)) can be used. Other relatedness analysis or quality control tools can also be used with the systems and methods of the present disclosure, as will be obvious to one skilled in the art upon the reading of the present specification.

### Artificial Neural Networks

A preferred method of memorizing the segmented data for future comparison following dimension reduction is using an artificial neural network (“ANN”). ANNs are nonlinear statistical data modeling tools where the complex relationships between inputs and outputs are modeled or patterns are found. As with non-linear dimension reduction techniques, artificial neural networks have seen considerable advances in scale and complexity. Artificial neural networks usually consist of multiple layers of interconnected compute units (neurons) that are inspired by biological neurons in the brain, and are ideally suited to complex classification tasks such as genetic population stratification (Bridges Met al., PLoS One, 6(5):e14802 (2011)).

An ANN has several advantages but one of the most recognized of these is the fact that it can actually learn from observing data sets. ANNs have the ability to utilize data samples rather than entire data sets to arrive at solutions, which saves both time and money. Neural nets are an abstraction of linear algebraic hyperplanes (i.e., neurons) that preserve information in receptive fields because of interconnectivity between neurons. The receptive fields of neural nets are very sensitive to localized information such as the features that contrast a dog image from a cat image and are inspired loosely by the mechanisms of biological neurons in the human brain. In some aspects of the present disclosure, a computer method is employed to facilitate extraction of the SNPs from the sequencing data. See, e.g., Li H et al., Bioinformatics. 2009 Aug. 15; 25(16): 2078-2079. The extraction of the information on nucleic acid sequences and genetic features can identify changes in the data based on, e.g., changes in the sequencing data from one, two, or an admixture of the cohorts used in the analysis, or as compared to a reference sequence as introduced to the ANN for the analysis.

Various forms of ANNs can be used with the present invention. Examples of artificial neural networks and their applications (e.g., in healthcare) can be found, e.g., in Baskin II et al., Expert Opin Drug Discov. 2016 August; 11(8):785-95 (2016); Hassabis D. et al., Neuron, 95(2):245-258 (2017).

For example, the invention can utilize a convolutional ANN or “CANN”. A CANN is a neural network created from a sequence of individual layers, with each successive layer operating on data generated by a previous layer.

In some examples, the convolutional artificial neural network system is configured by executing a backpropagation process based on the training data. In this way, the artificial neural network module executes a search for weight map parameters that best classify all of the training data. The design of the system's architecture may specify a number of parameters including a number of layers, a number of weight maps per layer, values of the weight maps, nature of the data extraction performed; whether contrast normalization is done.

In certain aspects, the systems of the disclosure include a standard neural network architecture, such as the architecture described by Krizhevsky, A et al., in “ImageNet Classification with Deep Convolutional Neural Networks,” NIPS, 2012, although any number of other neural network architectures can be used. See. E.g., Van Veen F, An Informative Chart, to Build Neural Network Cells, 2016; asimovinstitute.org; see also Visualizing and Understanding Convolutional Networks, European Conference on Computer Vision 2014, pp 818-833.

In some implementations, the feature data denoting the feature space output from the AI-based dimension reduction stage (i.e., coordinates in space, color, shape, depth, etc.) are converted into images (or other symbols) prior to input into the neural net classifier.

### Superpositions and Data Augmentation

To further improve the accuracy of the neural net, important spatial relationships or hierarchies between the segmented clusters may be taken into account to augment the training set. In this case, instead of classic transformation to achieve data augmentation, the non-linear dimension reduction parameter of perplexity can be employed to generate different frames containing different representations of the segmented data which could be used for data augmentation. Perplexity represents the balance between local and global features of the dimension reduced data. The present usage of perplexity to achieve data augmentation is hereby termed superposition as the neural net is forced to find coherence of identical input data that are in different positional states.

In this particular implementation, the frames generated by perplexity=30 and 40 were utilized as superposition inputs for the neural net because they represent a good middle ground where segmentation is retained but there are enough slight variations in the clusters' positions across frames to achieve data augmentation.

Additionally, instead of perplexity, neural net architectures that better account for important spatial relationships between objects (e.g., capsule nets (Hinton G, Sabour S, Frosst N. Matrix Capsules with EM Routing. Conference Paper at ICLR 2018. 2018)) could be successfully implemented to achieve superposition and thus better accuracy.

### Contour Plots to Examine Neural Net Activation

To gain mechanistic insight how the neural net is performing its memorization and identification function, an additional neural net can be constructed (e.g., nolearn/Lasagne/Theano stack or Scikit-learn) and employed to construct contour plots of neuronal activations to directly observe spatial receptive fields of output neurons. These can be overlaid onto 2D t-SNE plots for easy visualization and study of the mechanism that underpins the primary neural net's function, enabling garnering of insights that can in turn yield further optimization for improved performance.

### Population Stratification

Population stratification biases are an ongoing challenge in genetic association studies, including genome wide association studies. Population stratification occurs in the presence of undetected population structure whereby study samples comprising sets of individuals differ systematically in both genetic ancestry and the phenotype under investigation. Instead of identifying true association of alleles corresponding to disease phenotypes, spurious associations arise which can often be explained by differences in ancestry.

The most straightforward way to avoid population stratification biases is ensuring that the study sample is derived from a relatively genetically homogenous population. Of course, this is not always possible, and is the reason why statistical methodology needs to be applied to detecting and adjusting for population stratification. Genomic control aims to control for population stratification by first estimating the degree of inflation of the test statistics by comparing the median distribution of the test statistics for association as compared to that under the null (no association) distribution. Inflation of the test statistic can be the result of population stratification, cryptic relatedness between the samples, genotyping error, or be due to true association. The inflation is quantified in the form of the genomic inflation factor (λ), which is used to correct downward the test statistics by this factor under the assumption that the test statistics are equally inflated at each locus across the genome, which is not usually the case. A genomic inflation factor close to unity reflects no evidence of inflation, while values up to 1.10 are generally considered acceptable for GWAS. Another (preferred) method utilizes large samples and thousands of markers throughout the genome to estimate pairwise allele sharing between individuals (described above) and use the IBS matrix for all individuals to obtain a given number of principal components to adjust the effect estimates for population structure.

### Hardware Implementations

In certain implementations, the neural net architecture is translated to hardware, which is optionally on a system in support of a CPU. Such translation to hardware results in acceleration of the functions which can result in a significant increase in speed as compared to software implementations. For example, Artificial Intelligence (AI) Accelerators have been developed to emulate software neural nets on-chip. These stem from General Purpose Graphic Processing Units (GPGPUs) which because of their highly parallel nature, process millions of image representations more efficiently than CPUs and more closely resemble the massively parallel nature of biological neural nets. AI Accelerators extend on this by discarding traditional cannon of CPUs—for instance, removal of scalar values in IBM's TrueNorth Chip containing grids of 256 neural units (Merolla et al., Science, Vol. 345, Issue 6197, pp. 668-673 (2014)). This chip was recently used to generate spiking neural nets (Diehl PU et al., arXiv:1601.04187v1).

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. It should also be appreciated that the examples provide enabling guidance on the use of the combined features of the disclosure to apply such tools, systems and methods to other uses. The present aspects are, therefore, to be considered in all respects as illustrative and not restrictive.

The examples can be implemented in certain aspects by computers or other processing devices incorporating and/or running software, where the methods and features, software, and processors utilize specialized methods to analyze data.”

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

In the following examples, t-SNE was first applied on a hyper-dense, high-dimensional genetic representation of whole genome sequence datasets from individual humans. Following t-SNE dimension reduction, the resultant lower dimensional coordinates (e.g., 3D) of each data point were fed into an artificial neural network for training. The trained neural net was subsequently used to classify individuals according to percentage of heritage derived from each of the 26 world populations sampled in the 1000 Genomes Project (Genomes Project C, Auton A et al. Nature. 2015; 526(7571):68-74. Additional methods to improve accuracy (i.e., superpositions) or to undertand the mechanism underpinning the neural net's function (i.e., neural net to plot contour plots of neuronal activation) were also implemented.

### Example 1: Computational Platforms for Segregating Human Populations

A computational platform was constructed using genomics datasets and artificial intelligence analyses. Whole genome sequences were obtained from the 1000 Genomes Project phase 3 repository (Genomes Project C, Auton A et al., Nature. 2015; 526(7571):68-74. These consisted of DNA sequences of 2504 individuals sampled from 26 populations from around the world. Genetic variations amongst populations were derived from the GRCh37 reference genome. To conserve processing and storage resources, examination was limited to chromosome 1. Variant Call Format (VCF) files of variations were converted to bed files using pLink2 (Danecek P, et al., Bioinformatics.; 27(15):2156-8 (2011); Chang CC et al., GigaScience.4:7 (2015)).

These bed files had hyperdense chromosomal representations of ˜6 million SNPs and include all regions including exons and non-coding regions. The bed files were input into KING (Manichaikul A et al., Bioinformatics. 2010; 26(22):2867-73.278) to obtain pairwise coefficients of the relatedness of each individual to every other member of the set. A matrix composed of these KING coefficients was generated and used as input into the statistical tools t-distributed stochastic neighbor embedding (t-SNE) (van der Maaten L and Hinton G, Journal of Machine Learning Research, 9: 2579-2605 (2008)) and principal component analysis (PCA) as a comparator (Hotelling, H.; Journal of Educational Psychology, 24, 417-441, 49 8-520 (1933); Hotelling H. Biometrika, 28:3211-377 (193 6))

### Example 2: Nonlinear Dimension Reduction using t-SNE

The resulting KING map produced as set forth above in Example 1 was a high-level feature space composed of thousands of dimensions that are impossible to interpret with human eyes.

The unsupervised machine learning nonlinear dimension reduction technique t-SNE was used to further elucidate the clustering of individuals with specific SNPs. The parameters adopted were: perplexity=30, learning rate=200, early exaggeration=12 for 1000 iterations. For superpositions, one additional t-SNE frame was used as input into the neural net. The second input frame was generated at perplexity=40, while keeping other parameters constant. In addition, various number of output dimensions (2D to 6D) were tested. The segregation achieved by t-SNE was compared to the use of 2D PCA to segregate the same data set.

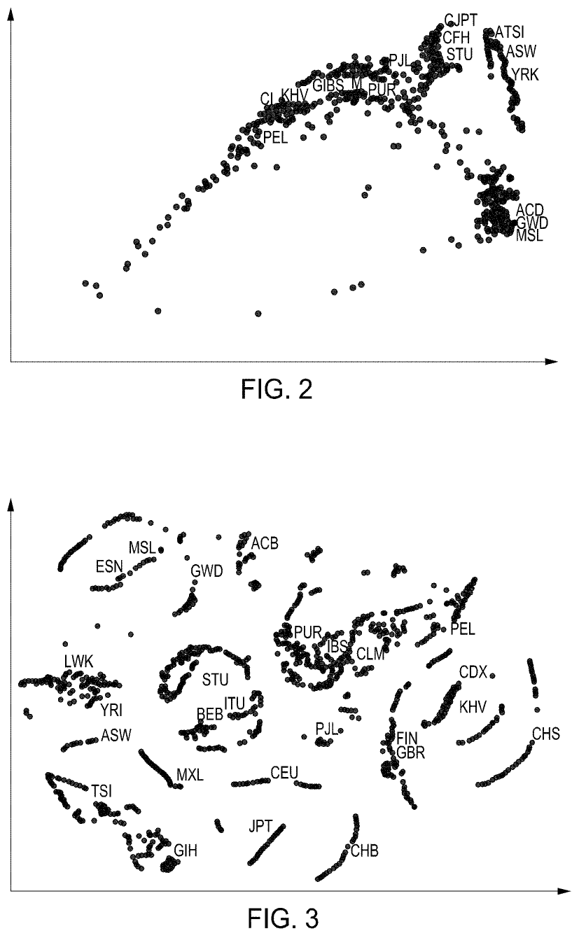

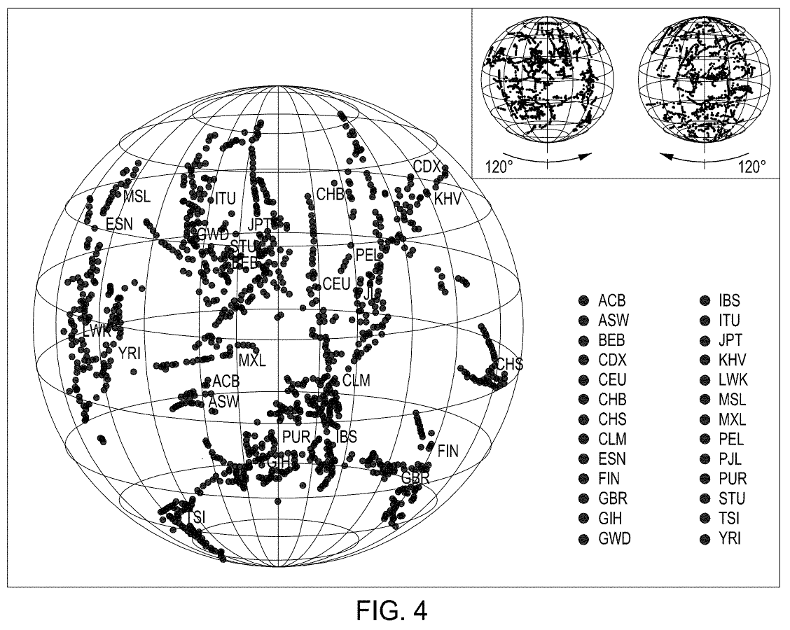

The results obtained showed that 2D PCA did not produce clear segmentation of the input data (FIG. 2). In contrast, the data input into t-SNE demonstrated clear clustering and separation of clusters (FIGS. 3 and 4). Labels were added post-hoc, and clear segregation of heritages was observed in 2D t-SNE (FIG. 3) and even more so with 3D t-SNE where clusters are distributed in the form of a sphere (FIG. 4). The visually tractable clustering of t-SNE was a deep representation as each point in the t-SNE map is an abstraction of ˜6 million SNPs. Further dimensional increases (e.g., 4D-6D) were also explored.

There were useful patterns within the visualized clustering that can be employed to draw valuable insights. For example, in 3D t-SNE, populations considered to be originator populations of more admixed population(s) (e.g., Luhya (LWK), Yoruba (YRI), British (GBR) and Finnish (FIN)), appear to segregate in the mantle near the surface of the sphere. Conversely, more recently admixed populations such as Puerto Rican (PUR) and Colombian (CLM), which are derived mainly from European, African and Native American populations, segregate near the core of the sphere (FIG. 4). In addition, each t-SNE iteration results in individuals moving towards a genetic equilibrium as the Kulback-Leibler distance is minimized. These movements can be captured as vectors that represent an additional feature for classification and deeper understanding of human genetic divergence.

### Example 3: Ancestry Classification using an Artificial Neural Network

Following dimension reduction using t-SNE, a deep neural network was constructed using 2D-6D t-SNE results as input to allow for the detailed comparison and identification of each individual according to the reported 26 heritages.

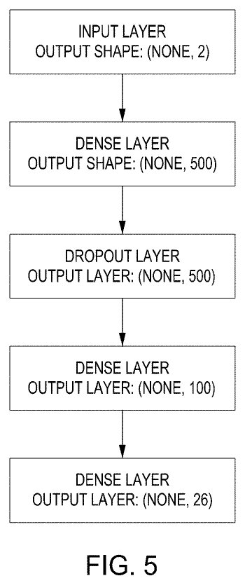

The output of t-SNE (i.e., the Cartesian coordinates of each individual in t-SNE space) was split into randomized training, test, and validation sets. The training set was used for supervised learning of a neural network. Analyses of segmentation was performed on the testing set and verified on the validation set. The neural network is a Theano-based stack with nolearn layered atop Lasagna, composed of 5 layers, including non-linear ReLU activations and a SoftMax output layer (FIG. 5). The input layer consisted of 2, 3, 4, 5 or 6 neurons for 2D, 3D, 4D, 5D or 6D t-SNE respectively (i.e., 2 neurons for x and y coordinates, 3 for x, y, and z, etc.). For superpositions, two frames of t-SNE were used as input into the neural net. Thus, the number of input neurons were doubled to 4, 6, 8, 10 or 12 neurons for 2D, 3D, 4D, 5D or 6D t-SNE respectively. The input layer is followed by 3 hidden layers composed of a dense layer of 500 neurons, a 50% dropout layer of 500 neurons, and a dense layer of 100 hidden neurons. These subsequently, upon training, output to 26 neurons, each sensitive to a single heritage from the 26 world populations in the 1000 Genomes Project dataset. Regularization was achieved mainly by dropout. All language was python within iPython interactive development environments hosted in the cloud or locally.

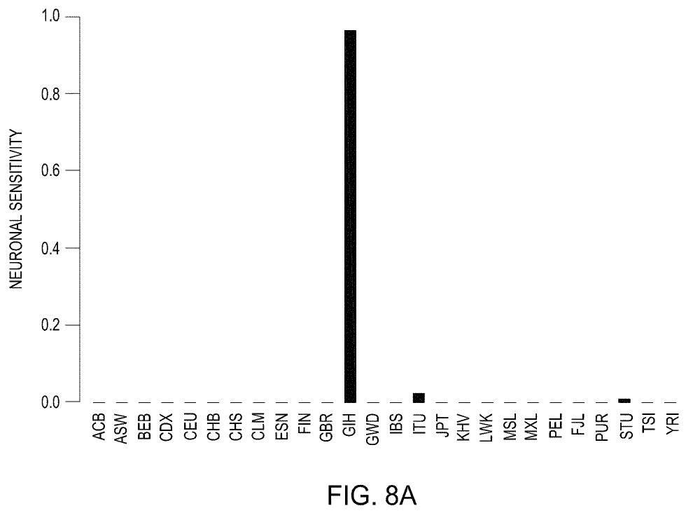

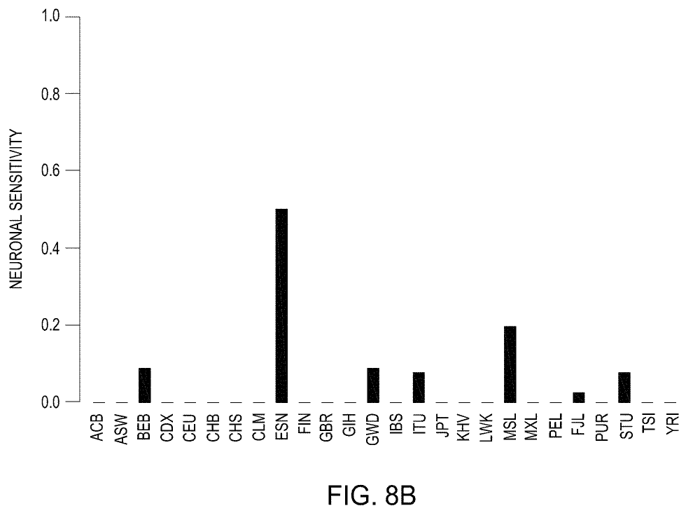

Histograms of the neuron firings indicate that each neuron is highly sensitive to a particular heritage FIGS. 8A and 8B. For some particular individuals, one neuron may fire at about 95% capacity while the sum of all the other neurons is about 5%, with most not firing at all (FIG. 8A). This indicated that an individual had low admixture. For many individuals, several neurons fired simultaneously, indicating multiple heritages, i.e., greater admixture (FIG. 8B). One of the three to five highest firing neurons was nearly always in agreement with the corresponding individual's self-described heritage. This neuron was not necessarily the highest firing neuron, suggesting the primary heritage of the individual was often not what was self-reported.

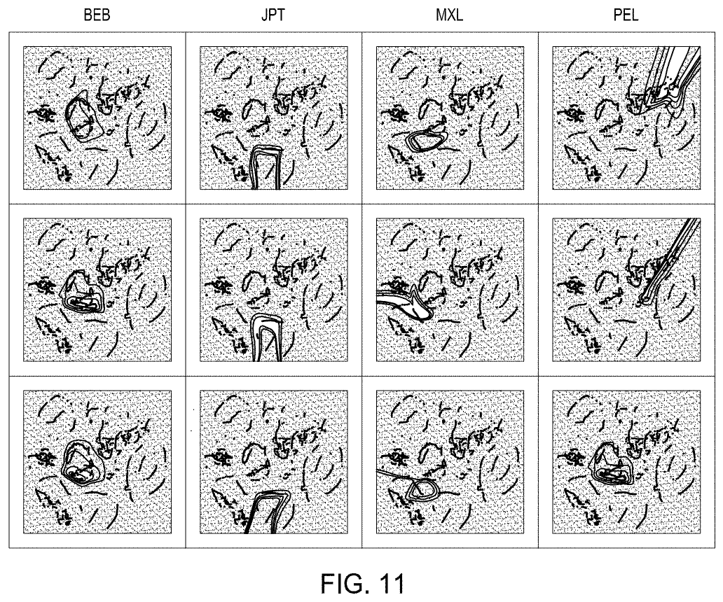

To gather mechanistic insight into how the neural net was performing its function, contour plots of neuronal activations were generated and overlaid onto 2D t-SNE plots for easy visualization (FIG. 11). Contour lines were plotted in 0.1 intervals from 0 to 1, and colored according to the ‘seismic’ colormap in Matplotlib (i.e. 0=Blue, 0.5=White, 1.0=Red). Each contour plot was specific for a single output neuron heritage (indicated by the column labels) and overlaid on the 2D t-SNE plot for visualization. Rows represent three separate runs of the neural net.

Results showed that for some heritages (e.g., JPT), the contour lines were close to each other and the corresponding output neuron fired at or close to 1, indicating detection of a tight cluster and highly confident classification by the neural net. Moreover, the contour pattern remained similar across multiple runs and enveloped the JPT cluster in 2D t-SNE space, indicating highly reproducible and accurate classification by the net. For MXL, the neural net performed classification well, but was less confident than JPT. This was indicated by the fact that in one of the three runs, the maximum activation of the MXL neuron was ˜50% compared to the other two runs, and the contour lines did not form a tight cluster. However, for the other two heritages tested (e.g. BEB and PEL), the neural net displayed some uncertainty in its classification (i.e., the contour shapes did not match across multiple runs).

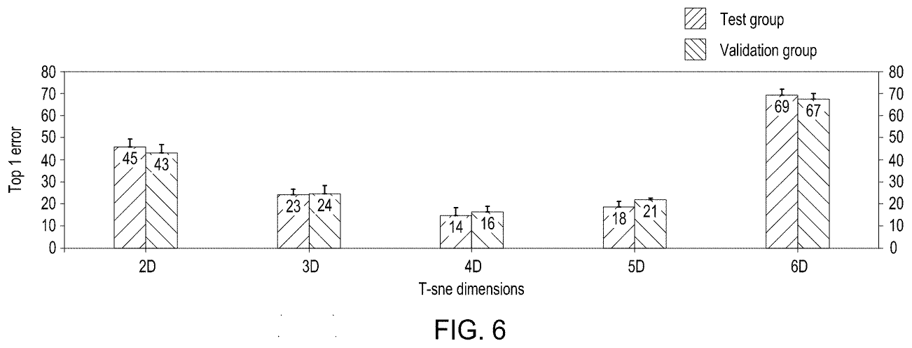

Next, the effect of different t-SNE dimensions on the performance of the neural net function was investigated (FIG. 6). For a more precise estimation of the neural net's accuracy, Top-n scores traditionally used in the machine learning community were adopted. For instance, Top-1 error is the likelihood that the ground truth label is not the top label predicted by the neural net (e.g., the individual claims to be Yoruba but the net's top firing neuron is not Yoruba).

Results showed that 4D t-SNE allowed for the best neural net performance (lowest top-1 error of ˜15%). However, further increases of the number of dimensions resulted in a drop in neural net classification performance, i.e., top-1 error of ˜20% and ˜68% for 5D and 6D t-SNE respectively (FIG. 6).

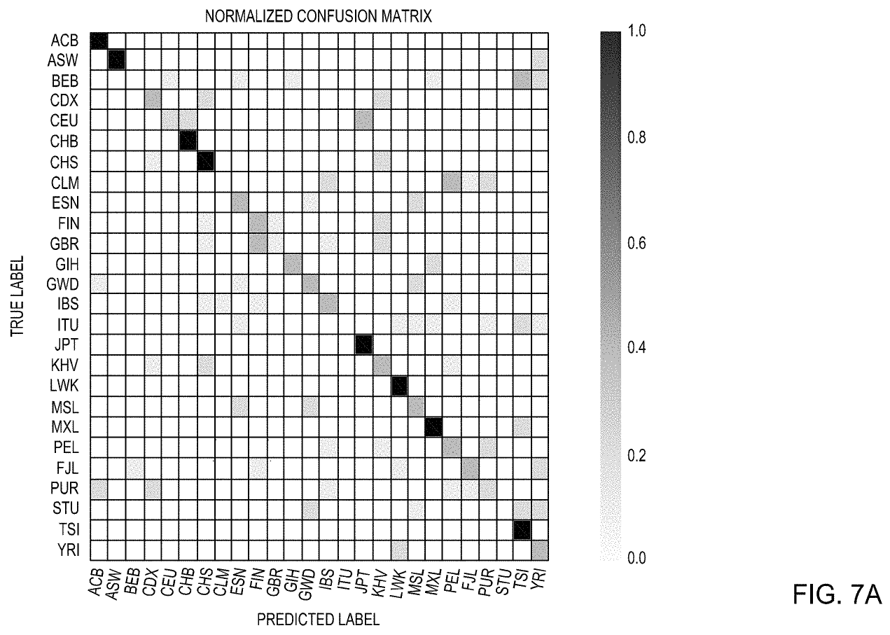

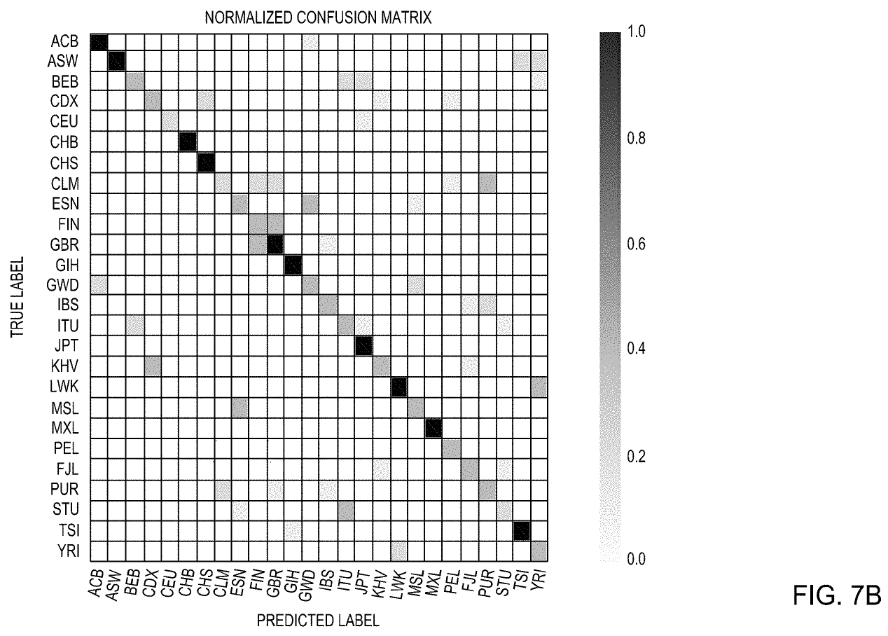

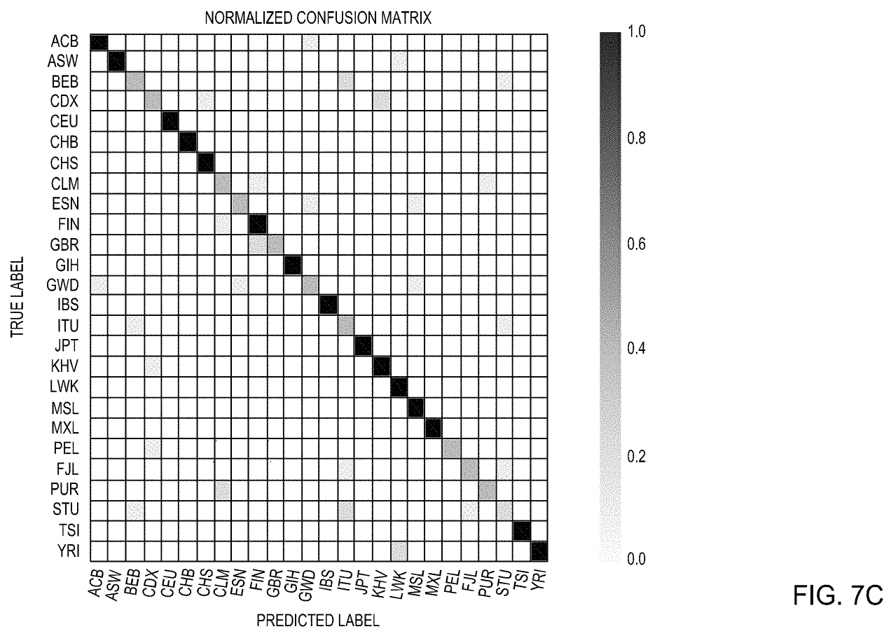

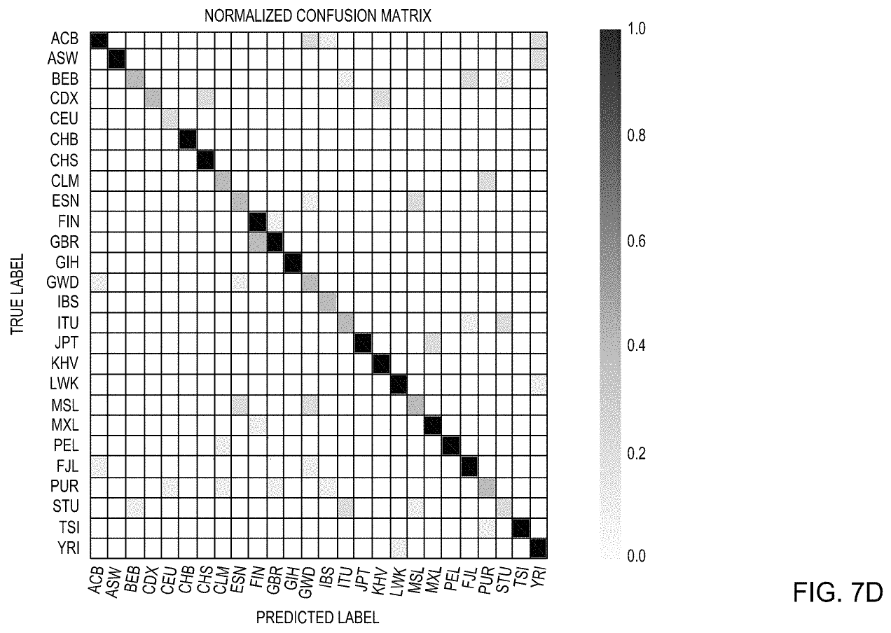

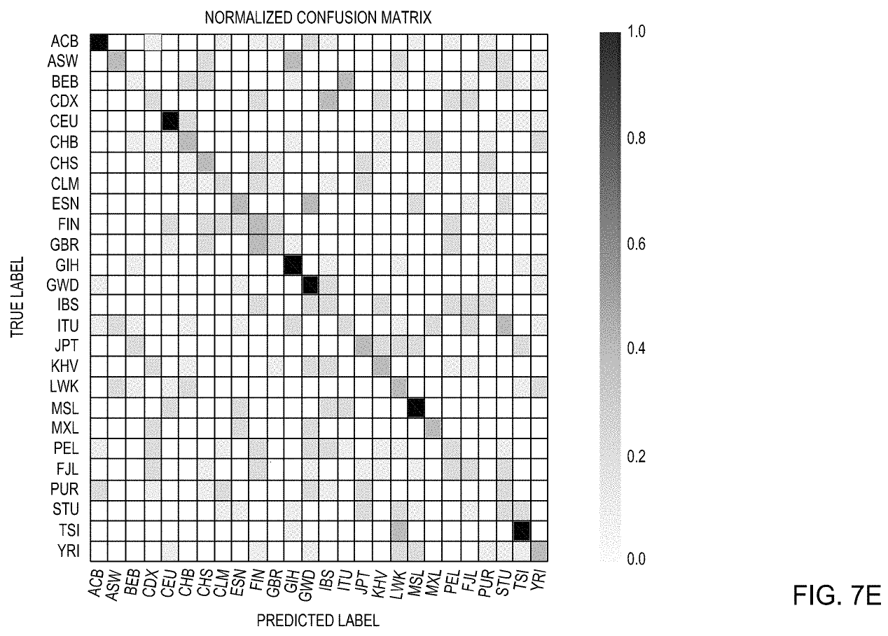

Confusion matrices of 2D through 6D t-SNE show that the net generally predicted these 26 labels well. FIG. 7A is the normalized confusion matrix for the 2D t-SNE prediction, FIG. 7B is the normalized confusion matrix for the 3D t-SNE prediction, FIG. 7C is the normalized confusion matrix for the 4D t-SNE prediction, FIG. 7D is the normalized confusion matrix for the 5D t-SNE prediction, and FIG. 7E is the normalized confusion matrix for the 6D t-SNE prediction. As shown in FIG. 7C, t-SNE with 4D demonstrated the best performance.

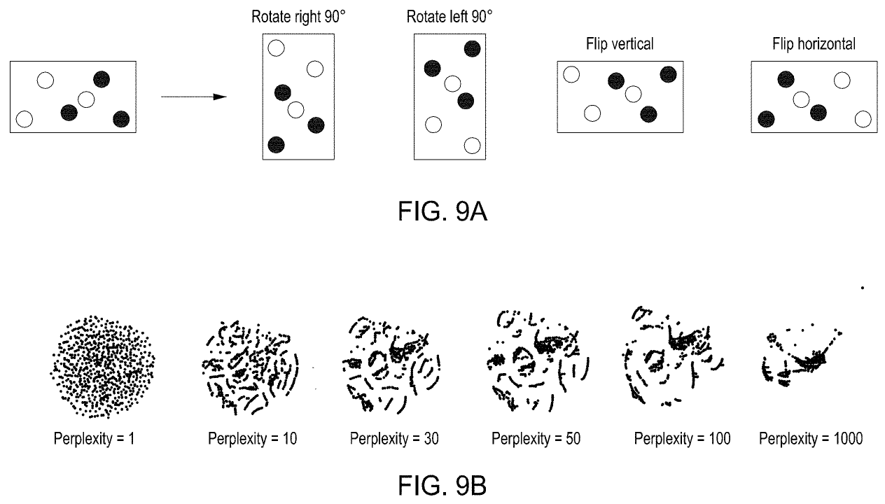

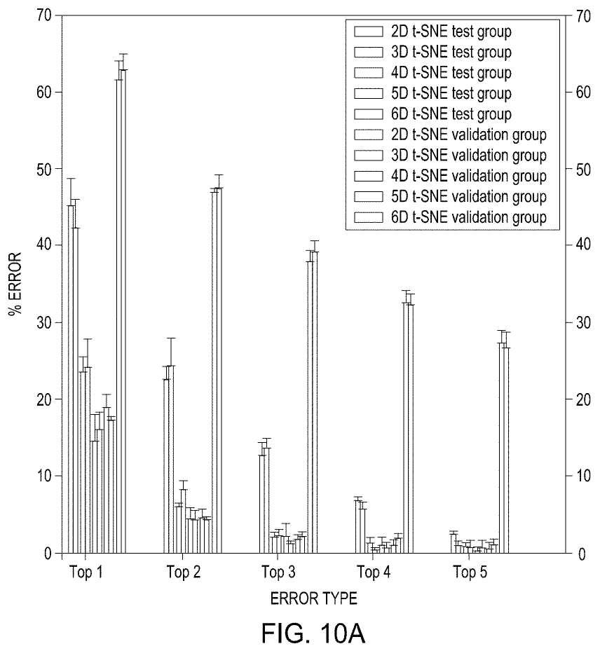

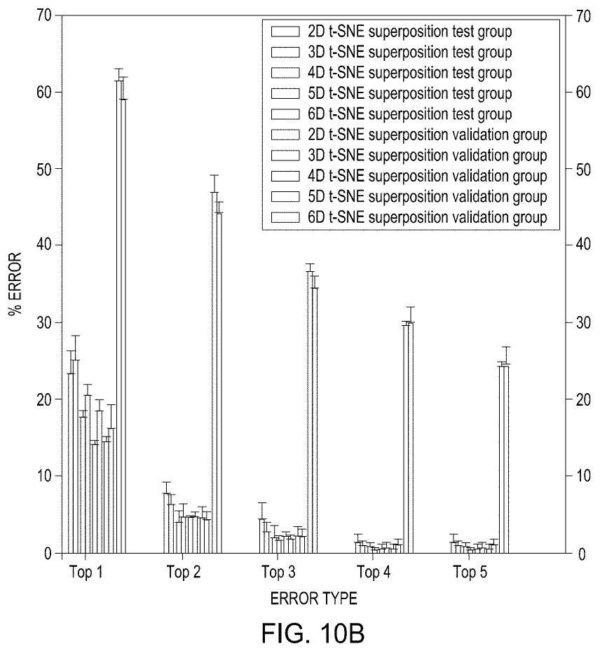

To further improve the neural net's performance, the superposition technique described above was leveraged (FIG. 9B) and top-1 to top-5 error scores for 2D to 6D t-SNE with superpositions were compared (FIG. 10B). Top-5 error is the likelihood that the correct label is not within the top five labels predicted by the neural net (e.g., subject claimed to be Yoruba and the net's top five predictions do not include Yoruba). The results of this particular implementation indicated that superpositions had the greatest impact on improving the accuracy of the neural net at low dimensions (2D t-SNE), decreasing top-1 and top-2 errors from ˜45% to ˜25% and ˜25% to ˜8% respectively (FIGS. 10A an 10B). In FIGS. 10A and 10B, the bars represent the average of n=3 passes of testing sets in the neural net; each testing set was randomized and contained 375 individuals. Error bars represent standard deviation.

These results show that it was possible to significantly improve the accuracy of neural net classification using inputs with certain dimension and/or superposition adjustments. Overall, the neural net's top-5 error rate is <0.5% for 4D t-SNE, suggesting that the net nearly always predicted the individuals' self-described label within its first five predictions, with comparable results at top-4.

### Example 4: Observation of Heritage Across Large Human Populations

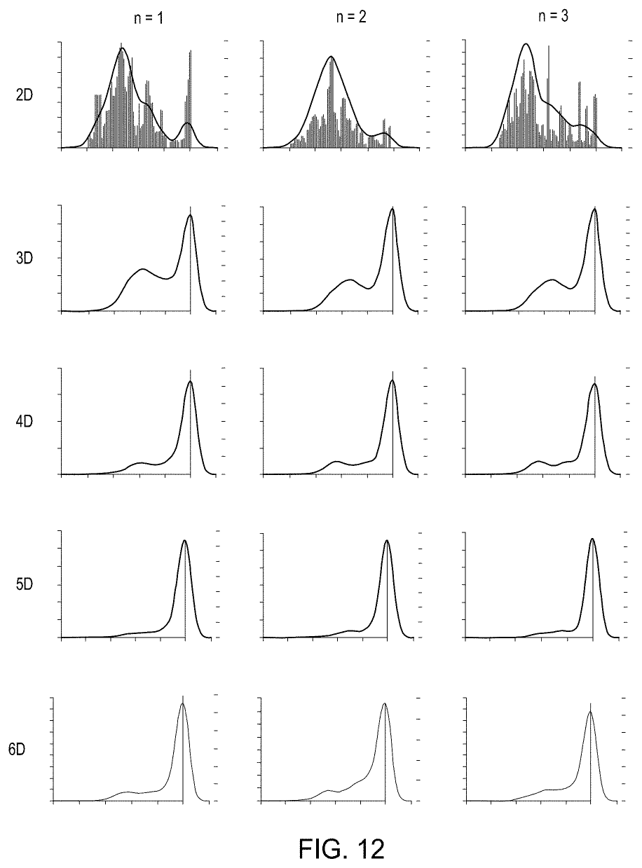

The activation intensity of neurons in the full 1000 Genomes Project population can reveal useful features of the high level genomic structure of Homo sapiens. One way of revealing these features through neuronal activation percentage was by plotting the population-wide data of frequency of occurrence of top firing neurons (y-axis) against the intensity at which they fired (x-axis). A trendline of the kernel density of this data is shown in FIG. 12. An individual of low ancestry admixture was expected to have a top firing neuron >90% activation representing one dominant heritage (see FIG. 8A). In contrast, a highly admixed individual was expected to have a top firing neuron of <50% activation with additional neurons firing at appreciable intensity across various heritages that comprise the component ancestries of the individual.

Many populations today are admixed but have clear origins in another region. For instance, African Americans have European as well as African heritages. A unimodal distribution of the highest-activation neuron (i.e., top firing neuron) per individual could indicate a smooth gene flow across populations. A bimodal distribution would indicate two distinct groups of populations. Tri-modal and multimodal distributions would suggest there are many bottlenecks to population-wide gene flow. For instance, geographical constraints of the six inhabited continents could result in six different distributions, each with its own pattern of admixture and originator profile.

Using the highest single neuron activation percentage per individual (i.e., top firing neuron), several regions of peak activity were found in the testing set. This data was dissected further by applying a kernel density function to obtain a curve representing the probability density estimate of the highest single neuron activation percentage across populations.

At 2D, the results showed a clear bimodal distribution of top neuronal activation intensity that was independently confirmed in the validation set (FIG. 12, top). The distribution was composed of a large component averaging 349 representative individuals per set and a small component averaging 29 individuals per set, respectively 92.3% and 7.7%. This evidence of a bimodal distribution of Homo sapiens provided support to the notion of a large population that had a greater range of admixture and a small population that was primarily composed of potential originators. However, at higher dimensions, the bimodal distribution shifted right so that the highly admix majority became the minority. (FIG. 12) This outcome was indicative of the neural net becoming increasingly more confident in its classification at higher dimensions (albeit wrongly at 6D given bad top-n error scores), stemming from more distant cluster separation in t-SNE space. With this higher confidence bordering on certainty in its primary choice at higher dimensions, the neural net ignored other heritage choices that are representative of admixture.

These results gave the first early evidence that contemporary Homo sapiens fit a bi-modal distribution with a large component of admixed individuals and a small component of less-admixed individuals. And although this picture reverses at higher t-SNE dimensions to favor the less-admixed group, this outcome was because the neural net became very confident in its primary choice to the exclusion of all secondary heritages, as evidenced by the contour plots and the fact that, at higher dimensions the neural net returned only the primary heritage of known recent admixed populations (e.g., ASW, PUR). As such, in this particular implementation, 2D t-SNE was found to be suitable for a detailed view of component heritages, whereas 4D t-SNE was preferable for primary heritage classifications.

While this invention is satisfied by aspects in many different forms, as described in detail in connection with the preferred invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. §112, ¶6.

What is claimed is:

1. A system for data segmentation and identification, comprising:
a) a first tool for segregation of complex data sets; and
b) a second tool for memorization of segregated data and/or identification of new data by comparison to the memorized data.

2. The system of claim 1, wherein the segregation of complex data sets comprises non-linear dimension reduction.

3. The system of claim 2, wherein the non-linear dimension reduction is AI-based.

4. The system of claim 3, wherein the AI-based non-linear dimension reduction comprises t-distributed stochastic neighbor embedding (“t-SNE”)

5. The system of claim 1, wherein the system further comprises a data correction tool.

6. The system of claim 1, wherein the tool for memorization of segregated data and identification of new data by comparison to the memorized data is AI-based.

7. The system of claim 1, wherein the tool for memorization of segregated data and identification of new data by comparison to the memorized data comprises an artificial neural net.

8. A method for identification of data from one or more individuals, comprising:
a) inputting a plurality of data points into a tool for nonlinear dimension reduction;
b) applying nonlinear dimension reduction to the data points to segment the data into clusters;
c) inputting the segmented data of step b). into an artificial intelligence (“AI”)-based tool;
d) inputting one or more individual data points into the AI-based tool comprising the segmented data;
e) comparing the data from the one or more individual data points to the segmented, clustered data; and
f) identifying the one or more individual data points by correlation with the segmented data memorized within the AI-based tool.

9. The method of claim 8, wherein the non-linear dimension reduction is AI-based.

10. The method of claim 9, wherein the AI-based non-linear dimension reduction comprises t-distributed stochastic neighbor embedding (“t-SNE”)

11. The method of claim 8, wherein the method further comprises data correction of the plurality of data points.

12. The method of claim 8, wherein the memorization of segregated data and identification of new data by comparison to the memorized data utilizes an artificial neural net.

13. The method of claim 8, wherein the plurality of data points are genetic data used for population stratification.

14. The method of claim 8, wherein the plurality of data points are used to determine the presence and/or concentration of microbial organisms or virus.

15. The method of claim 8, wherein the plurality of data points are used to determine the genetic heritage of one or more individuals.

16. The method of claim 8, wherein the plurality of data points are used to determine the predicted response of one or more individuals to a particular therapeutic intervention.

17. The method of claim 8, wherein the plurality of data points are used to determine the presence and/or stage of a disease in one or more individuals.

18. The method of claim 8, wherein the plurality of data points are used to determine the genetic features associated with a phenotype.

19. The method of claim 8, wherein different frames containing different representations of the segmented data are employed to increase the system's performance.

20. A method for including or excluding one or more individuals as being descended from a particular heritage, comprising the steps of:
a) inputting data from the genetic data of a plurality of individuals into a tool for nonlinear dimension reduction;
b) applying nonlinear dimension reduction to the data from a plurality of individuals to segment the data into clusters;
c) inputting the segmented genetic data of step b). into an artificial intelligence (“AI”)-based tool;
d) inputting genetic data from one or more individuals into the AI-based tool comprising the segmented genetic data;
e) comparing the genetic data from one or more individuals to the segmented genetic data from the plurality of individuals; and
f) including or excluding one or more individuals as being descended from a particular heritage by identifying the correlation of the individual data with segmented genetic data within the AI-based tool.
